(19) United States
(12) Patent Application Publication
(10) Pub. No.: US 2023/0034547 A1
(43) Pub. Date: Feb. 2, 2023

(54) MULTI-FUNCTION SANITIZING LAMP ASSEMBLY

(71) Applicant: OttLite Technologies, Inc., Tampa, FL (US)

(72) Inventors: John Sheppard, Tampa, FL (US); Louise Gardner, Tampa, FL (US); Kelly Martelli, Tampa, FL (US); Jessica Kremkau, Tampa, FL (US)

(21) Appl. No.: 17/873,726

(22) Filed: Jul. 26, 2022

Related U.S. Application Data
(60) Provisional application No. 63/203,595, filed on Jul. 27, 2021.

(51) Int. Cl. A61L 9/20

(52) U.S. Cl. CPC A61L 9/20; A61L 2209/14; A61L 2209/11; A61L 2209/15

(57) ABSTRACT

A lamp assembly having a light source and a sanitizing system. The assembly can be formed of various differing configurations to correspondingly provide one or more systems for sanitizing the surrounding air. Some of the applicable sanitizing systems are configured to be adaptable to provide differing levels of performance in order to provide the warranted sanitizing effectiveness in efficient manners when using the assembly.

### RELATED APPLICATIONS

This application claims the benefit of U.S. Provisional Patent Application No. 63/203,595 filed Jul. 27, 2021, and the entire content of that application is incorporated herein by reference.

### FIELD OF INVENTION

The present invention relates to lighting apparatus, and more particularly, to lighting apparatus configured with air sanitizing systems.

### BACKGROUND

Lighting apparatus can be grouped into two basic groups: adjustable and non-adjustable. Some smaller-scale lamps are made to be adjustable; however, this is not the case for all. As is known, many smaller-scale lamps are designed for use on tables, most notably desks. In most cases, such lamps are configured to be easily portable. Consequently, such lamps can be moved closer to areas where light is needed, e.g., working or reading areas. However, if not configured to ably direct light as warranted, the lamps can be found quite inefficient, i.e., the amount of light emitted from the lamp being a much higher percentage than the amount that can be effectively used by a user.

The reason for the inefficiency of such smaller non-adjustable lamps is that they are often designed to provide concentric distribution, and result in a generalized type of lighting for a circular area surrounding the lamp. This type of light distribution often involves the lamp having a generally vertical construction that often is aesthetically pleasing to the onlooker. However, as described above, such lamp design does a rather poor job of collectively directing the light for a person working at, or proximate to the lamp, because a significant portion of the light generated from the lamp is not aptly directed for the person's use. Therefore, while such non-adjustable lamps have a desirable aesthetic appearance, they often lack in efficiency. On the other hand, adjustable lamps typically have one or more portions thereof which can be oriented to direct the light from the lamp there from. As a result, they would be considered more efficient with respect to collective use of their generated light.

In recent years, some lamp assemblies have been designed to exhibit both lighting and sanitizing functions. For example, UV (Ultra-Violet) light, generally in the range between 400 nm and 100 nm can be used for disinfecting air, water, and surfaces. Such UV wavelengths can break down molecular bonds within DNA, which kills or inactivates microorganisms. The specific bands of UV wavelengths for effectively killing various types of organisms have been extensively studied and are known. For example, the wavelength of about 254 nm has been determined to be optimal for killing common microorganisms found in water and air, and the wavelength of 293 nm is optimal for killing certain other types of microorganisms. These optimal wavelengths are in the UV-B range (315 nm-280 nm) and UV-C range (280 nm-100 nm).

As described above, adjustable lamp designs enable light therefrom to be selectively directed, so as to be efficiently used. If also incorporating sanitizing systems, such lamp designs can provide not only a well-lit area, but also a sanitized working space for the user. However, skilled artisans would appreciate that non-adjustable lamps can also provide well-lit and sanitized areas, yet without as much focus on particular areas as can be achieved with adjustable lamps. There are many techniques and configurations by which sanitizing can be provided. To that end, lighting efficiency can sometimes be sacrificed for sanitizing effectiveness, and vice versa, simply based on size of the lamp assembly. Given this, lamp designers and manufacturers are often left in a bit quandary when deciding on combinations for lighting and sanitizing components in keeping the overall design effective and efficient in its performance, while also relatively compact. The present invention addresses these issues.

### SUMMARY

Certain embodiments of the present invention involve a lamp assembly having a light source and a sanitizing system. The assembly can be formed of various differing configurations to correspondingly provide one or more systems for sanitizing the surrounding air, which includes the general environment and objects/surfaces therein. Some of the applicable sanitizing systems are configured to be adaptable to provide differing levels of performance in order to provide the warranted sanitizing effectiveness in efficient manners when using the assembly.

In one embodiment, a lamp assembly is provided. The lamp assembly includes a base section and a shroud section for holding a light source for the lamp assembly. Each of the base section and the shroud section forms a sanitizing system. Each system provides a different level of sanitizing effect on surrounding air in the environment. The systems are selectively controllable to be used independently or in combination.

In another embodiment, a lamp assembly is provided. The lamp assembly includes a base section and a shroud section for holding a light source for the lamp assembly. The base section forms a sanitizing system. The sanitizing system includes a UV (Ultra-Violet) source and a fan. The base section houses the UV source and the fan so as to be situated between vents defined on opposing front and rear surfaces of the base section. The UV source for emitting wavelengths of light across the UV-C and UV-B ranges. The base section sized for the UV source and fan for providing an airflow rate through the system.

In a further embodiment, a lamp assembly is provided. The lamp assembly includes a base section and a shroud section for holding a light source for the lamp assembly. Each of the base section and the shroud section forms a sanitizing system. Each system provides a different level of sanitizing effect on surrounding air in the environment. The sanitizing system of the base section includes a UV (Ultra-Violet) source, a filter, and a fan. The fan when actuated creates flow of air through the filter and in proximity of the UV source prior to being exhausted from the assembly. The sanitizing system of the shroud section includes the light source, wherein the light source emits visible light yet at wavelength of 405 nm.

### DESCRIPTION OF THE DRAWINGS

FIG. 1 is a front perspective view of a lamp assembly in accordance with certain embodiments of the invention.

FIG. 2 is a rear perspective view of the lamp assembly of FIG. 1.

FIG. 3 is a front perspective of another lamp assembly in accordance with certain embodiments of the invention.

FIG. 4 is a rear perspective view of the lamp assembly of FIG. 3.

FIG. 5 is an exploded assembly view of an airflow system in accordance with certain embodiments of the invention.

### DETAILED DESCRIPTION

The following detailed description should be read with reference to the drawings, in which like elements in different drawings are numbered identically. The drawings depict selected embodiments and are not intended to limit the scope of the invention. It will be understood that embodiments shown in the drawings and described below are merely for illustrative purposes, and not intended to limit the scope of the invention as defined in the claims.

In the description herein, the term “lamp assembly” is used generally to represent lighting apparatus that is designed to work in conjunction with a light source. While lamp assemblies embodied herein may most notably be associated with table or desk lamps, the invention should not be limited thereto. Instead, the embodiments of the invention described herein are just as applicable to wall- or floor-mounted lamps. Likewise, the term “light source” is used herein to generally represent any source of visible light that can be used with the lamp assembly. For example, the light source can involve any means designed to emit visible light there from, including those sources using an outer bulb envelope. As alluded to above, it should be understood that description of these few examples are not meant to limit the uses of the embodied lamp assembly, but conversely to provide a sense of these and other potential applications that the present invention avails itself to.

In general, the adjustable lamp assembly of the present invention as embodied herein involves a plurality of operably-coupled sections that function together, allowing the light source of the assembly when used to be selectively adjusted, or angled, e.g., between 0 and 90 degrees, from horizontal. While it should be appreciated that designs embodied herein are also applicable to non-adjustable lamp assemblies, enabling the design to be adjustable enables further versatility, such that the output can be directed as desired. To that end, when using such lamp assembly, the assembly can be selectively adjusted between a generally protruding profile (e.g., falling between 0 and 60 degrees from horizontal) and a generally streamlined profile (e.g., falling between 60 and 90 degrees from horizontal).

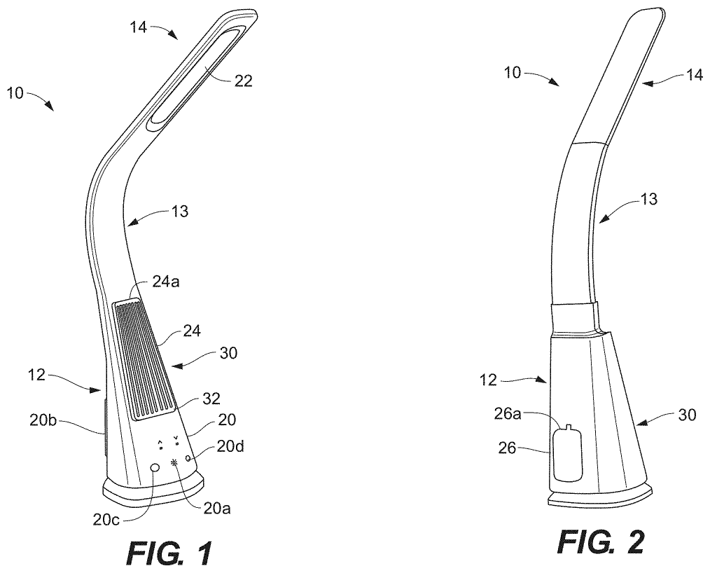

FIG. 1 is a front perspective view of a lamp assembly in an angled configuration in accordance with certain embodiments of the invention, while FIG. 2 is a rear view of the lamp assembly in differing angled configuration. As shown, the lamp assembly 10 includes a base section 12, an elbow section 13, and a shroud section 14, with the sections 12, 13, 14 being operatively joined. In certain embodiments, the base section 12 and shroud section 14 are formed of ABS plastic; however, the sections 12, 14 could be alternately formed of other materials. Conversely, the elbow section 13 is formed of a material (such as braided steel or thermoformed plastic) configured to be angularly adjusted yet sufficiently rigid to enable the section 13 to hold its adjusted position after being angled for its warranted use. In certain embodiments, the elbow section 13 can have first and second end segments 16, 18 (hidden from view in FIGS. 1 and 2, but exemplarily represented with dashed lines in FIG. 4) to be snuggly received within corresponding ends of the base and shroud sections 12, 14. In some cases, the end segments 16, 18 can have couplings 16a, 16b having a larger outer diameter than the end segments 16, 18, providing a formed fit with corresponding internal catches of the base section 12 and the shroud section 14. With such coupling, a housing for the lamp assembly 10 can be formed with substantially-smooth outer surfaces extending from the base section 12, along the elbow section 13, and to the shroud section 14. In certain alternate embodiments, the base section 12 or shroud section 14 could be formed of one or more portions to include the elbow section 13, while the remaining end 16 or 18 of the elbow section 13 can be snuggly received by the corresponding base section 12 or shroud section 14 as described above.

As described above, the base section 12 and the shroud section 14 are operatively joined via intermediary, the elbow section 13, which serves as the adjustable portion of the assembly 10. To illustrate such adjustability, reference is made to FIGS. 1 and 2, in which the shroud section 14 is shown at differing angles from horizontal. For example, the angle of the shroud section 14 from horizontal shown in FIG. 1 is between about 45 and 60 degrees, while the angle from horizontal shown in FIG. 2 is between about 60 and 75 degrees. As described above, the elbow section 13 can be tubing, formed of a material configured to be angularly adjusted yet sufficiently rigid to enable the section 13 to hold its adjusted position after being angled. Such material, in certain embodiments, is braided steel; however, the section 13 could be alternately formed of other like-performing materials.

While not visibly depicted, mechanisms for powering the lamp assembly 10 are included and incorporated in the assembly 10. To that end, in certain embodiments, the assembly 10 is powered wirelessly, e.g., through batteries or other power storage means. However, providing power to the lamp assembly 10 in this fashion should not limit the invention. For example, in certain alternate embodiments, the assembly 10 can have an electrical cord feeding into the base section 12 for providing a source of electric power. Regardless of power source (and while not visibly shown), electrical continuity would extend from such source up to a control panel 20, exemplarily located on the base section 12 for clear view and accessibility for the user. In certain embodiments, there are a plurality of buttons on the panel 20 for the differing mechanisms of the assembly 10, as will be further described herein. For example, relative to a light source 22 of the assembly 10 (e.g., recessed in shroud section 14), a power button 20a can be provided on the panel 20. More will be described of the light source 22 herein, but for now, its powering and control are described. The button 20a can function as a single pole switch; however, such switch could be further configured as desired. For example, the button 20a can be further electrically tied to one or more of dimming and/or timing functionalities, the configuration of which would be known to the skilled artisan. In certain embodiments, buttons 20b for adjustment of such dimming and/or timing functions can be further provided on the control panel 20.

Electrical continuity would further internally extend from the light source power button 20a up to the light source 22 of the lamp assembly 10. In certain embodiments, the assembly 10 is configured to electrically accept the light source 22 within its shroud section 14. For example, the shroud section 14 can include a recessed electrical socket, with the light source 22 to be accepted therein. While not being shown, in certain embodiments, the light source can involve an LED light source. To that end, the LED light source can involve a single LED or a plurality of LEDS provided in one or more strips. Of course, in some embodiments, the light source could involve a combination of LED and another light source type, which, instead of being powered directly through an electric feed, may in some cases need to be initially manipulated, e.g., by ballast means, such as for fluorescent lighting sources. As would be understood, such initial manipulation means could be integral to the light source, but it may also need to be remote from the light source. In such cases, such means could further be held within (e.g., within the base section 12), or remote to, the lamp assembly 10.

As described above, some lamp assembly designs combine both lighting and sanitizing functions. However, relative to smaller-scale lamp assemblies (such as for use at tables and desks), it is often a challenge to provide the warranted lighting and sanitizing functionalities, while keeping the overall design relatively compact. As alluded to above, there are many techniques and configurations by which sanitizing can be provided. For example, in certain embodiments, the lamp assembly 10 is designed to have one or more systems to provide sanitizing function. In certain embodiments, a first sanitizing system 30 is configured with the base section 12. With reference to FIGS. 1 and 2, the system 30 is largely concealed within the base section 12. This enables the lamp assembly 10 to maintain its aesthetic appearance, but also forms a protective shield relative to the sanitizing process. Particularly, the sanitizing system 30 involves a UV source 32 (e.g., LED) configured to emit a wavelength across either the UV-C or the UV-B ranges, and preferably somewhere between 260 and 290 nm, so as to be optimal for destroying the DNA of (and thereby killing) particularly resistant microorganisms in the air. In certain embodiments, the UV source 32 emission has a wavelength of about 280 nm. The same powering mechanism used to power the light source 22 is further used to power the UV source 32 of the first sanitizing system 30. Providing electrical connection for the UV source 32, so as to bring power to the source 32, is known in the art, and thus not further detailed herein. in certain embodiments, the control panel 20 has a power button 20c for activation of the UV source 32.

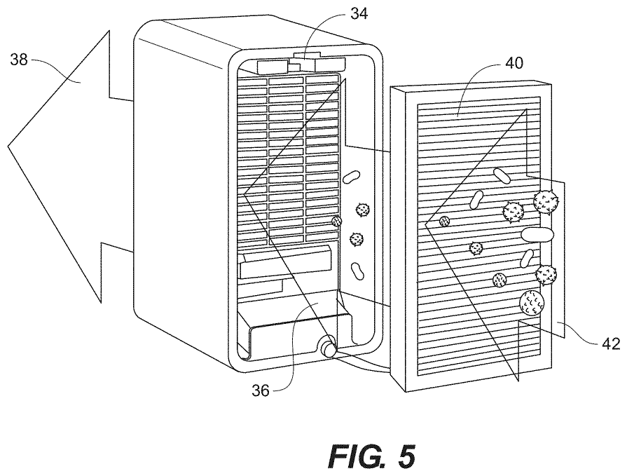

As shown via FIGS. 1 and 2, there are front and rear vents 24, 26 shown in the lamp assembly 10. As further illustrated, each of these vents 24, 26 have corresponding grates 24a, 26a at the corresponding front and rear outer surfaces of the base section 12. These grates 24a, 26a can be used to help maintain an aesthetic appeal for the lamp assembly 10, yet also to screen off the UV source 32 from users. As noted, when emitting a wavelength across the UV-C and UV-B ranges, preferably between 260 and 290 nm, the UV source 32 can be effective in killing particularly resistant microorganisms in the surrounding air. However, periods of exposure to such UV wavelengths can also be harmful to humans. Thus, the source 32 is concealed within the base section 12. Accordingly, to be effective for sanitizing, flow of air must be created through the base section 12 and in the vicinity of the UV source 32. Accordingly, the sanitizing system 30 includes a fan 34 (not visibly shown in FIGS. 1 and 2 yet depicted in FIG. 5). The fan 34 is positioned between the front and rear grates 24a, 26a and, as such, is concealed within the base section 12. Providing electrical connection for the fan 34, so as to bring power to the source 34, is known in the art, and thus not further detailed herein. In certain embodiments, the fan 34 has a corresponding power button 20d for its activation on the control panel 20.

With reference to FIGS. 1, 2, and 5, activation of the fan 34 enables air to flow from rear to front of the assembly 10, or more particularly in through the grate 26a at rear vent 26, through the fan 34 and in proximity of the UV source 32, and out through the grate 24a at front vent 24. To that end, the rear vent 26 serves as the air intake, while the front vent 24 serves as the air exhaust, with the exhausted air being in sanitized form. It should be appreciated that the kin 34, based on its size and rate of rotation, has a corresponding rate of air passage there through (e.g., cubic feet per minute), which also corresponds with the rate of sanitizing, CADR (clean air delivery rate), for the sanitizing system 30. In certain embodiments, as described below, the fan airflow rate can be 7 cubic feet per minute or 9 cubic feet per minute or 13 cubic feet per minute, depending on the warranted sanitating parameters. To that end, in certain embodiments, the flow rate may range from 7 cubic feet per minute to 20 cubic feet per minute, and in more preferable embodiments, the flow rate may range from 8 cubic feet per minute to 13 cubic feet per minute.

Of course, a primary consideration for any sanitizing system is whether its sanitizing rate is sufficient for its purpose (i.e., for sanitizing the room in which the system is to be used). To that end, the size of the room in which the lamp assembly 10 is to be used and the quantity of air changes per hour (ACH) desired for the room, need to both be factored in determining what CADR is needed from the sanitizing system 30 (and whether the system 30 can provide the desired degree of air sanitizing). Regarding ACH (again, air changes per hour), a generally accepted sanitizing rate is considered a minimum of 2 ACH (for regular air cleaning). Alternately, for greater degree of cleaning (if occupants of the room are generally susceptible, such as those with air-borne allergies and asthma), a minimum of 5 ACH may be viewed as more acceptable, while for heavily contaminated air, a minimum of 8 ACH may be recommended. To that end, the higher the ACH, the more rapid the air cleaning takes place in that room.

Considering a room measuring 6′ in length, 6′ in width, and 8′ in height (288 cubic feet), and looking to minimally achieve 2 ACH for the room, the CADR for the system 30 would need to be 9 cubic feet per minute. To that end, the base section 12 has capacity to house a fan rated for such performance. By way of comparison, for a room measuring 10′ in length, 10′ in width, and 8′ in height (800 cubic feet), and looking to minimally achieve 2 ACH for the room, the CADR for the system 30 would need to be 13 cubic feet per minute. Again, the base section 12 has capacity to house a fan rated for such performance. In summary, the larger the enclosed room, the higher the CADR (again, the clean air delivery rate) is needed for the sanitizing system 30. Likewise, the higher the desired ACH (again, air changes per hour) for the room, the higher the CADR (again, the clean air delivery rate) is needed for the sanitizing system 30. In each of these cases dictating higher CADR, there is higher performance correspondingly warranted from the fan 34, which translates to larger size and/or increased rate of rotation to enhance the rate air passage there through. To that end, the base section 12 is sized to accommodate fans to achieve the above-exemplified CADR ratings for the system 30, as well as larger-sized fans to achieve even higher CADR ratings.

Shifting back to FIG. 5, as noted above, the fan 34 is exemplarily shown. As depicted, there is an incoming flow of air 36 (which, with reference back to FIG. 2, would correlate with the airflow into the rear vent 26 and then) into the fan 34. Likewise, there is an outgoing flow of air 38 (which, with reference back to FIG. 1, would correlate with the airflow out the front vent 24 and) from the fan 34. With reference back to the assembly 10 of FIG. 1, the flow of air is directed past the UV source 32 to sanitize the air. In certain embodiments, the UV source 32 is positioned between the fan 34 and the front grate 24a; however, the UV source could also be positioned between the fan 34 and the rear grate 26a.

The UV source 32 represents a first level of sanitizing of the sanitizing system 30. In certain embodiments, as shown in FIG. 5, a second level of sanitizing is provided for the system 30 via a filter 40. With reference to FIGS. 2 and 5, the filter 40 is positioned between the fan 34 and the rear grate 26a, which in such cases, corresponds with the UV source 32 being positioned on the opposing side of the fan 34. The filter 40 is provided to perform an initial sanitizing (filtering) of the incoming flow of air relative to some of its larger particulates 42. In certain embodiments, the filter 40 is a HEPA (high-efficiency particulate air) filter, which are known to trap particulates generally having diameter of 0.3 um and greater. As is known, HEPA filters are graded by efficiency level. To that end, the higher the grade, the better the filter. For example, HEPA filters, grades 10 through 12 (H10-H12), trap 85-99.5% of particles having diameter of 0.1 um. By way of comparison, HEPA filters, grades 13 and 14 (H13 and H14), trap 99.9% of such particle. In certain embodiments, the HEPA filter can be a H10 type; however, it should be appreciated that this can be varied depending on application. For example, other smaller-size particulates, such as bacteria and microorganisms, can also become trapped in the filter via their being tied to larger-sized particles, such as pollen, dirt, and dust. It should be appreciated that including a filter 40 in the sanitizing system 30 may affect airflow through the fan 34. Particularly if the filter 40 has high efficiency, the fan 34 may need to be pushed harder to achieve the desired CADR rating, which translates to larger size and/or increased rate of rotation for the fan to enhance the rate of air passage there through. As described above, the base section 12 has the capacity to accommodate larger-sized fans as warranted.

As a brief recap, the lamp assembly 10 includes light source 22 and one or more systems to provide sanitizing function. As described above, in certain embodiments, the assembly 10 includes one sanitizing system 30. The system 30, in turn, can include one level of sanitizing function, via UV source 32 or via filter 40, or via both to facilitate two levels of sanitizing function. In certain embodiments, a second sanitizing system 50 is configured with the shroud section 14. For example, and with reference to FIGS. 1 and 2, the light source 22 in the shroud section 12 forms the sanitizing system 50. To that end, and like the UV source 32 (e.g., LED source) of the first sanitizing system 30, the light source 22 used in providing visible light from the lamp assembly 10 can further provide sanitizing function. For example, UV light, generally in the range between 400 nm and 100 nm, can be used for disinfecting air, water, and surfaces. To that end, UV light greater than 400 nm is in the visible spectrum and such wavelengths are considered safe. Accordingly, the light source 22 emits at wavelengths of greater than 400 nm, and more preferably close to 400 nm to have greater sanitizing function. In certain embodiments, the light source 22 is an LED with emission at wavelength of 405 nm. The 405 nm wavelength is well known in the lighting industry, and its uses/advantages are covered in U.S. Pat. Nos. 9,039,966 and 9,839,706 the teachings of which are incorporated herein for reference. To achieve certain levels for lighting and sanitizing performance, in certain embodiments, the wavelength would be maintained at minimum irradiance level of 0.5 W/cm².

With the above serving as a backdrop, it should be appreciated that in certain applications, the sanitizing activity of the lamp assembly 10 can be viewed as significant as its lighting function. To that end, in the pursuit of combining these functionalities, the assembly 10 is not only efficiently conceived (in terms of the assembly's general size), but benchmarks for effectiveness are achievable as well. For example, with reference to the lamp assembly 10 as described herein, a kill rate percentage of 99.9% has been achieved for certain microorganisms, such as Methicillin Resistant Staphyloccus Aureus (MRSA) and Escherichia Coli. In addition, a kill rate of 99.9% of MS2 bacteriophage has also been achieved, which correspondingly enhances the assembly's application, given that MS2 is widely known to be used as a surrogate for pathogenic RNA viruses, including COVID-19.

Achieving a particular kill rate via the lamp assembly 10, such as 99.9%, can involve use of one or more of the sanitizing systems of the assembly 10, namely the system for the base section 12, the system for the shroud section 14, or both. Relative to the base section 12, the UV (LED) source 32, the fan 34, and the filter 40 are each variable elements, size/wattage and quantity of the light source 32, size and rate of rotation of the fan 34, and size and grade of the filter 40. As already described, a change in one of the elements can directly impact another of the elements. For example, an increase in grade of the filter 40 (which helps trap greater extents/degrees of microorganisms) generally corresponds with the fan 34 needing to be of larger size in order to pub air through the assembly base section 12 (and correspondingly, the filter 40). However, if the grade of filter 40 is reduced, the size of fan 34 can also be of smaller size/power; however, there would likely be greater passage of microorganisms through the filter 40, which, if a high kill rate is needed, the size/quantity of UV source 32 may need to be enhanced. In certain embodiments, the sanitizing system of the base section 12 can be used to effectively kill MS2 bacteriophage.

Shifting to the shroud section 14 of the assembly 10, the light source 22 can be solely used for sanitizing function. As already described, the light source 22 can emits at wavelengths of greater than 400 nm, and more preferably close to 400 nm to have greater sanitizing function. In certain embodiments, the light source 22 is an LED with emission at wavelength of 405 nm, and in certain embodiments, maintained at minimum irradiance level of 0.5 mW/cm². In certain embodiments, the sanitizing system of the base section 12 can be used to effectively kill MRSA and Escherichia Coli.

Continuing with the above, along with the kill rate of the lamp assembly 10, there is further variable that is often tracked, namely the time it takes to achieve such kill rate within a specific area. For example, it is often informative to indicate on product packaging how long it takes for the product to reduce an amount of microorganisms (e.g., MS2 bacteriophage) in a room by a certain percentage, such as 99.9%. Much like rate, time is closely linked to parameters of the UV (LED) source 32, fan 34, and filter 40 relative to the sanitizing system of the base section 12. Each of these elements can be enhanced, as exemplified above; however, if the elements are to be provided in a lamp assembly package (such as lamp assembly 10), the aesthetics of the assembly can play a part in the commercial success of such product. Thus, the sizes of one or more of the UV (LED) source 32, fan 34, and filter 40 can be correspondingly limited. Regarding the shroud section 14, its light source can still be equipped with LED with emission at wavelength of 405 nm to achieve similar results, i.e., reducing an amount of microorganisms (e.g., MRSA and Escherichia Coli) in a room by a certain percentage, such as 99.9%.

Thus, the lamp assembly 10 can be provided to involve multi-system and multi-level sanitizing functionality. In one embodiment, the assembly 10 includes first and second sanitizing systems 30 and 50, with the first system 30 formed of two layers of sanitizing function and the second system 50 formed of one layer of sanitizing function. To that end, the assembly 10 can be used in a variety of manners, depending on the specific application (e.g., what degree of sanitizing is warranted). For example, in one case, perhaps no light is needed from the assembly 10, yet high degree of sanitizing is required. For such application, the buttons 20c (for the UV source 32) and 20d (for the fan 34) are actuated on the control panel 20. Alternately, perhaps light is needed, with moderate degree of sanitizing. In turn, the buttons 20a (for the UV light source 22) and 20d (for the fan 34) are actuated on the control panel 20. In other cases, the button 20a may be solely actuated when light and a minimal degree of sanitizing is needed (e.g., just at the lit workspace).

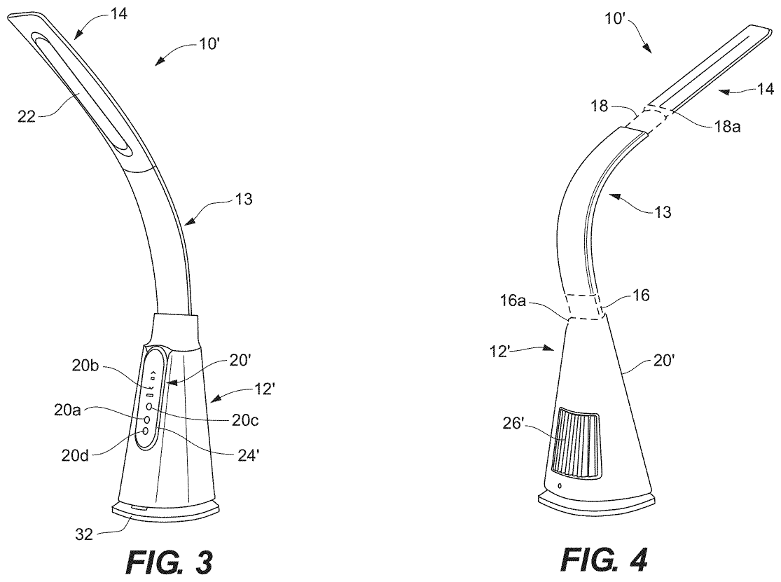

Turning to FIGS. 3 and 4, another lamp assembly 10′ is shown, similar to the lamp assembly 10 of FIGS. 1 and 2. To that end, the functioning of the lamp assembly 10′ is similar to what has already been described relative to the lamp assembly 10. However, the venting and control panel 20′ relative to the base section 12′ are different. Particularly, the control panel 20′ is positioned atop of the front vent 24′. Such design still permits airflow through rear vent 26′ and out from front vent 24′, yet around the periphery of the control panel 20′, providing a more aesthetic appeal for the consumer on front surface of the lamp assembly 10′.

It will be appreciated the embodiments of the present invention can take many forms. The true essence and spirit of these embodiments of the invention are defined in the appended claims, and it is not intended the embodiment of the invention presented herein should limit the scope thereof.

What is claimed is:

1. A lamp assembly, comprising
a base section; and
a shroud section for holding a light source for the lamp assembly, each of the base section and the shroud section forming a sanitizing system, each system providing a different level of sanitizing effect on surrounding air in the environment, the systems selectively controllable to be used independently or in combination.

2. The lamp assembly of claim 1 further comprising an elbow section operably joining the base section to the shroud section, the elbow section comprising tubing.

3. The lamp assembly of claim 2 wherein the tubing is formed of a material having flexible rigidity, such that the shroud section can be angularly adjusted relative to the base section and then maintained in such adjusted position.

4. The lamp assembly of claim 1 wherein the sanitizing system of the base section comprises a UV (Ultra-Violet) source and a fan, the base section housing the UV source and the fan so as to be situated between vents defined on opposing front and rear surfaces of the base section, the UV source for emitting a wavelength across the UV-C and UV-B ranges.

5. The lamp assembly of claim 4 wherein the UV source comprises one or more LEDS configured to emit at wavelengths of between about 260 nm and about 290 nm.

6. The lamp assembly of claim 4 wherein the fan when actuated creates flow of air through the rear vent, in proximity of the UV source, and out through the front vent.

7. The lamp assembly of claim 6, further comprising a filter housed by the base section positioned between the fan and the rear vent, wherein the sanitizing system of the base section comprises two levels of sanitizing effect, the first level from the UV source and the second level from the filter.

8. The lamp assembly of claim 7, wherein the filter is positioned to perform an initial sanitizing of the air flowing through the rear vent, wherein the UV source is positioned between the fan and front vent.

9. The lamp assembly of claim 4 further comprising a control panel, wherein the UV source and fan are actuated via a corresponding button on the panel.

10. The lamp assembly of claim 9 wherein the panel is located on the base section, atop the front vent.

11. The lamp assembly of claim 7 wherein the sanitizing system of the shroud section comprises the light source, wherein the light source emits visible light yet at wavelengths close to 400 nm.

12. The lamp assembly of claim 11, wherein the light source comprises one or more LEDS configured to emit at wavelength of 405 nm.

13. A lamp assembly, comprising
a base section; and
a shroud section for holding a light source for the lamp assembly, the base section forming a sanitizing system, the sanitizing system comprising a UV (Ultra-Violet) source and a fan, the base section housing the UV source and the fan so as to be situated between vents defined on opposing front and rear surfaces of the base section, the UV source for emitting a wavelength across the UV-C and UV-B ranges, the base section sized for the UV source and fan for providing an airflow rate through the system.

14. The lamp assembly of claim 13 wherein the UV source comprises one or more LEDS configured to emit at wavelengths of between about 260 nm and about 290 nm.

15. The lamp assembly of claim 13 wherein the fan when actuated creates flow of air through the rear vent, in proximity of the UV source, and out through the front vent.

16. The lamp assembly of claim 15, further comprising a filter housed by the base section and positioned between the fan and the rear vent, wherein the sanitizing system of the base section comprises two levels of sanitizing effect, the first level from the UV source and the second level from the filter.

17. The lamp assembly of claim 16, wherein the filter is positioned to perform an initial sanitizing of the air flowing through the rear vent, wherein the UV source is positioned between the fan and front vent.

18. The lamp assembly of claim 16 further comprising a sanitizing system for the shroud section and comprising the light source, wherein the light source emits visible light yet at wavelengths close to 400 nm.

19. The lamp assembly of claim 18, wherein the light source comprises one or more LEDS configured to emit at wavelength of 405 nm.

20. A lamp assembly, comprising
a base section; and
a shroud section for holding a light source for the lamp assembly, each of the base section and the shroud section forming a sanitizing system, each system providing a different level of sanitizing effect on surrounding air in the environment;
wherein the sanitizing system of the base section comprises a UV (Ultra-Violet) source, a filter, and a fan, the fan when actuated creates flow of air through the filter and in proximity of the UV source prior to being exhausted from the assembly; and
wherein the sanitizing system of the shroud section comprises the light source, wherein the light source emits visible light yet at wavelength of 405 nm.
